(19) United States
(12) Patent Application Publication
(10) Pub. No.: US 2025/0312116 A1
(43) Pub. Date: Oct. 9, 2025

(54) REUSABLE SURGICAL INSTRUMENT FOR MINIMALLY INVASIVE PROCEDURES

(71) Applicant: Distalmotion SA, Epalinges (CH)

(72) Inventor: Ricardo Daniel Rita BEIRA, Lausanne (CH)

(21) Appl. No.: 18/942,292

(22) Filed: Nov. 8, 2024

Related U.S. Application Data
(63) Continuation of application No. 18/048,794, filed on Oct. 21, 2022, now Pat. No. 12,262,969.
Application No. 18/048,794 is a continuation of application No. 16/870,870, filed on May 8, 2020, now Pat. No. 11,478,315.
Application No. 16/870,870 is a continuation of application No. 15/536,576, filed on Jun. 15, 2017, now Pat. No. 10,646,294.
(60) Provisional application No. 62/094,080, filed on Dec. 19, 2014.

(51) Int. Cl. A61B 34/37; A61B 17/29; A61B 34/00; A61B 34/30; A61B 90/00

(52) U.S. Cl. CPC A61B 34/37; A61B 17/29; A61B 34/71; A61B 2017/2901; A61B 2017/2927; A61B 2017/2948; A61B 2034/305; A61B 2090/0813

(57) ABSTRACT

Disclosed is a reusable surgical instrument (1) with an articulated end-effector (3), such as a dissector, scissor or grasper, to enhance a surgeon's performance during various surgical procedures. The longitudinal axis of the instrument is defined by a shaft (2), comprising an internal structural element (2″) covered by an external tube (2′), which may be inserted through a surgical incision into the body of a patient, optionally through a trocar. The articulated end-effector (3) is mounted on the distal extremity of the shaft's internal structural element and comprises a plurality of links interconnected by a plurality of joints, whose movements are remotely actuated by the surgeon's hands. This remote actuation is accomplished via mechanical transmission (5, 6, 7), mainly composed of flexible elements, which are able to deliver motion from a set of actuation elements, placed at a proximal extremity of the shaft (2), to the instrument's articulated end-effector (3). The external tube (2′) can be easily and individually detached from the shaft (2) after each procedure, so that the instrument (1) can be more effectively cleaned and sterilized.

### FIELD OF THE INVENTION

The present invention relates to the field of minimally invasive articulated instruments such as graspers, dissectors, and scissors, wherein the orientation of the distal end effector in relation to the instrument shaft is able to be controlled. More particularly, the invention relates to reusable surgical instruments that have to be cleaned and sterilized after each procedure. Most specifically, the invention relates to such instruments wherein the actuation and orientation of the distal end-effector is remotely performed, from the proximal to the distal extremity of the instrument shaft, by mechanical transmission elements. The instrument of the present invention is intended to be used primarily in surgical procedures, wherein instruments with articulated end-effectors are passed through incisions or trocars into a patient's body cavity, which may be optionally inflated with insufflation gas.

### BACKGROUND OF THE INVENTION

Open surgery is still the standard technique for most surgical procedures. It has been used by the medical community for several decades and consists of performing the surgical tasks by a long incision in the abdomen or other body cavity, through which traditional surgical tools are inserted. However, due to the long incision, this approach is extremely invasive for the patient, resulting in substantial blood loss during the surgery and long and painful recovery periods in an in-patient setting.

In order to reduce the invasiveness of open surgery, laparoscopy, a minimally invasive technique, was developed. Instead of a single long incision, one or more smaller incisions are made in the patient through which long and thin surgical instruments and endoscopic cameras are inserted. Because of the low degree of invasiveness, laparoscopic techniques reduce blood loss and pain while also shortening hospital stays. When performed by experienced surgeons, these techniques can attain clinical outcomes similar to open surgery. However, despite the above-mentioned advantages, laparoscopy requires advanced surgical skills to manipulate the rigid and long instrumentation through small incisions in the patient.

Traditionally, laparoscopic instruments, such as graspers, dissectors, scissors and other tools, have been mounted on straight shafts. These shafts are inserted through small incisions into the patient's body and, because of that, their range of motion inside the body is reduced. The entry incision acts as a point of rotation, decreasing the freedom of the surgeon for positioning and orienting the instruments inside the patient. Therefore, due to the drawbacks of its instrumentation, laparoscopic procedures are mainly limited to use in simple surgeries, while only a small minority of surgeons is able to use them in complex procedures. Therefore, there has been a clear trend for providing distal articulations to end-effector elements of laparoscopic instruments, allowing the distal effector elements to be angulated with respect to the longitudinal axis of the instrument shaft.

Laparoscopic instruments can be provided as disposable or reusable medical devices. Disposable devices are thrown away after each utilization, without having the need to be cleaned. On the other hand, reusable devices must be cleaned and sterilized after each procedure. In many instances, cost-effectiveness and operating room efficiency requires that instruments be cleaned, sterilized and re-used.

Although techniques such as steam sterilization have been widely used, they are often insufficient to reach all of the blood and tissue residues that can enter a surgical instrument during a surgical procedure. In particular, for the case of instruments with articulated end-effectors (like the one disclosed in U.S. Pat. No. 7,819,894), the cleaning and sterilization processes are even more challenging. The higher mechanical complexity of the articulated end-effector brings additional places where tissue and blood can easily infiltrate. In addition, in order to be airtight and keep the body cavity inflated, these systems are constructed with an elongated and closed tubular body, from where the penetrated blood and tissue are very difficult to be removed.

Some reusable laparoscopic instruments (like to ones disclosed in EP1889579, U.S. Pat. Nos. 5,147,357, 5,304,203, 5,308,358, 5,368,606, 5,603,723 and US20090299141) can be disassembled for cleaning and thereafter reassembled for subsequent utilization. This enables access to the interior portions of the instrument tube and the internal mechanical elements housed therein, which results in more reliable cleaning and sterilization methods. However, this solution has only been used in instruments with low complexity end-effectors (mainly with a single distal degree of freedom), where the assembly and disassembly procedures are relatively simple and can therefore be easily accomplished by the hospital staff. This easy assembly/disassembly procedure cannot be applied to existing articulated instruments (like the one disclosed in U.S. Pat. No. 7,819,894). Indeed, in these instruments, the external tube of the instrument's shaft has the double function of giving structure to the instrument shaft and providing a sealing function for the instrument with respect to the trocar in order to preserve the inflation of the abdominal cavity where the instrument is operated. This limitation in the design of existing articulated instruments makes it impossible to remove the outer tube, which poses a significant challenge for the cleaning and sterilization of such instruments. Without being able to remove the outer tube, direct access to the internal elements of the articulated instrument is not possible, meaning that cleaning tools cannot be directly applied to the elements requiring cleaning, despite the fact that blood and tissue may have contaminated these elements during a surgical procedure. While some articulated instruments allow for the passage of a stream of water as a method of cleaning the internal elements, this does not provide for complete cleaning and is not an efficient solution.

Accordingly, an aim of the present invention is to overcome the aforementioned drawbacks of known devices by providing a new surgical instrument with an articulated end-effector, with uses in a cable-driven surgical instrument, where the external tube composing the instrument's shaft can be easily removed, for efficient cleaning, and subsequently reassembled for utilization.

### SUMMARY OF THE INVENTION

Theses aims and other advantages are achieved by a new articulated and reusable surgical instrument in the form of, for example, a dissector, scissor or grasper. The instrument comprises an articulated end-effector, placed at the distal extremity of an instrument shaft, which comprises an internal structural element and an external tube. The shaft defines the longitudinal axis of the instrument and is able to move according to the mobility constraints imposed by a body incision, which includes a rotational movement about its own axis. This rotation also causes the rotation of the end-effector, mounted on the distal extremity of the shaft. Thus, the instrument shaft has the triple function of (1) positioning the end-effector within the interior of the patient's body, (2) allowing the passage of the different transmission elements that are able to actuate the different distal end-effector articulations and (3) avoiding the passage of air through the instrument, in order to maintain the inflation of the body cavity where the instrument is operating. While the two first functions are achieved by the internal structural element, the third function is primarily achieved by the external tube. Since its primary function is not mechanical, the external tube can be easily and individually detached from the instrument after each procedure. This enables proper access to the internal elements passing through and disposed on the shaft so that the instrument can be more effectively cleaned and sterilized. Finally, the external tube can be easily re-attached to the instrument for the next usage.

With the above mentioned features, this reusable instrument can combine the performance benefits of highly articulated instruments with the benefits of most simple laparoscopic instrumentation, which can be easily and almost completely assembled and disassembled by the hospital staff so that internal components can be accessed for a more effective cleaning and sterilization. This results in a unique combination of safety and performance that is currently not available.

### BRIEF DESCRIPTION OF THE FIGURES

The invention will be better understood according to the following detailed description of several embodiments with reference to the attached drawings, in which:

FIG. 1 shows a perspective view of a reusable surgical instrument according to an embodiment of the invention;

FIG. 2 shows a perspective view of a reusable surgical instrument according to an embodiment of the present invention with a schematic cutout of the external tube of the instrument shaft, through which is it possible to see the internal structural elements passing through the instrument shaft;

FIG. 3 shows a perspective view of a reusable surgical instrument according to an embodiment of the present invention with a schematic cutout of the external tube of the instrument shaft, through which is it possible to see different mechanical transmission elements;

FIG. 4 shows a perspective view of an articulated end-effector of a reusable surgical instrument according to an embodiment of the invention;

FIG. 5 shows an articulated end-effector according to an embodiment of the present invention in a first active position;

FIG. 6 shows an articulated end-effector according to an embodiment of the present invention in a second active position;

FIG. 7 shows an articulated end-effector according to an embodiment of the present invention in a third active position;

FIG. 8 shows an articulated end-effector according to an embodiment of the present invention in a fourth active position;

FIG. 9 shows an articulated end-effector according to an embodiment of the present invention in a fifth active position;

FIG. 10 shows actuation topology for a first distal end-effector link according to an embodiment of the present invention;

FIG. 11 shows actuation topology for a second distal end-effector link according to an embodiment of the present invention;

FIG. 12 shows actuation topology for a proximal end-effector link according to an embodiment of the present invention;

FIG. 13 shows a perspective view of proximal hub with different proximal rotating elements according to an embodiment of the present invention;

FIG. 14 shows a simplified path of a flexible transmission element actuating a distal articulation of an end-effector according to an embodiment of the present invention;

FIG. 15 shows a procedure through which an external tube of an instrument shaft can be assembled and disassembled on a reusable surgical instrument according to an embodiment of the present invention;

FIG. 16 shows a detailed perspective view of sealing and transversal elements mounted on an internal structural element according to an embodiment of the present invention.

### DETAILED DESCRIPTION OF THE INVENTION

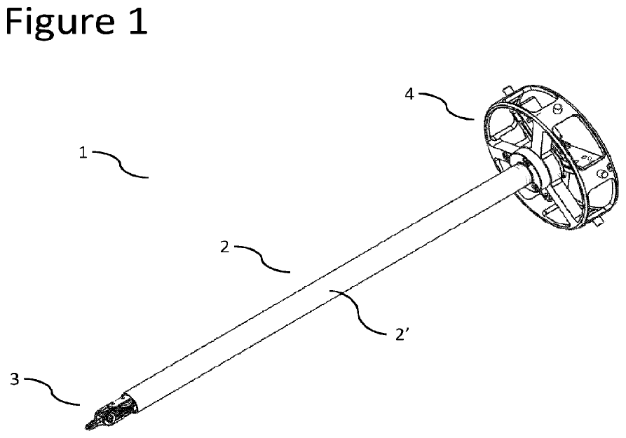

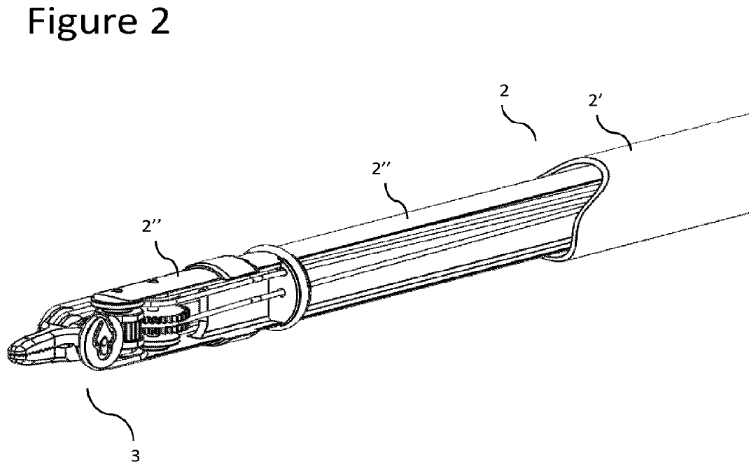

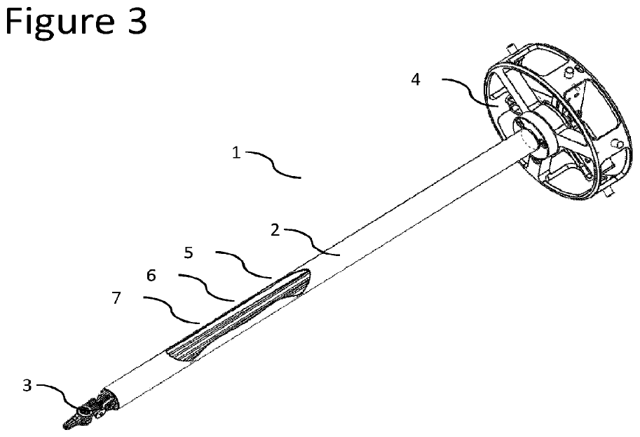

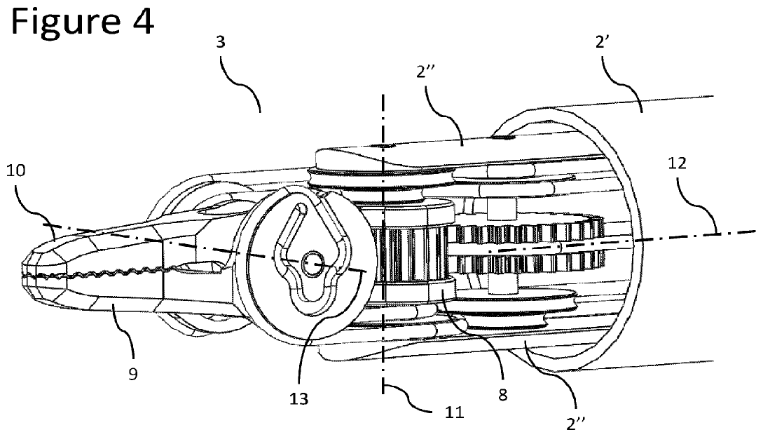

A reusable surgical instrument 1 for minimally invasive surgical procedures, with a detachable external tube 2′, constructed in accordance with an embodiment of the present invention, is described herein, and is seen generally in FIG. 1. This instrument 1 includes a main shaft 2, a distal articulated end-effector 3 and a proximal hub 4. Referring to FIG. 2, the shaft 2 is composed of two different elements: an internal structural element 2″ and an external tube 2′. The internal structural element 2″ provides a stable positioning to the end-effector 3 and to allow the passage of the different mechanical elements 5, 6, 7 that are able to deliver motion to the different end-effector links 8, 9, 10 from the proximal hub 4 at the proximal extremity of the instrument (FIGS. 3 and 4). The external tube 2′ protects the internal elements on the shaft 2 when passing through the incision and avoids the passage of air through the instrument 1, in order to maintain the inflation of the body cavity where the instrument 1 is operating.

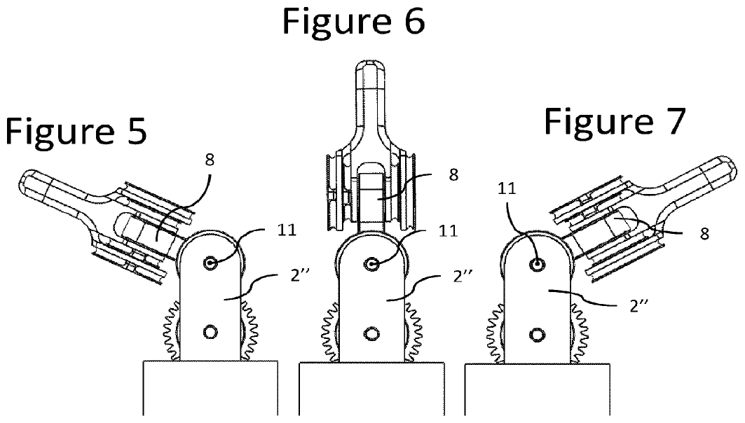

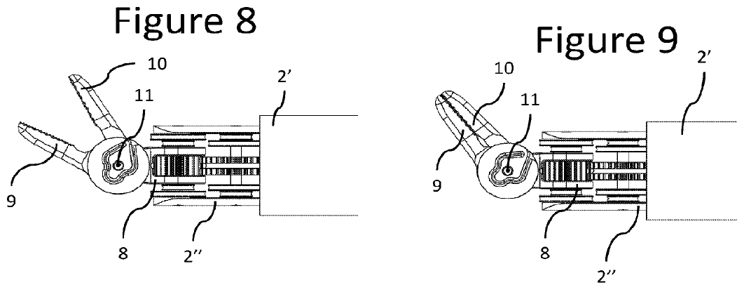

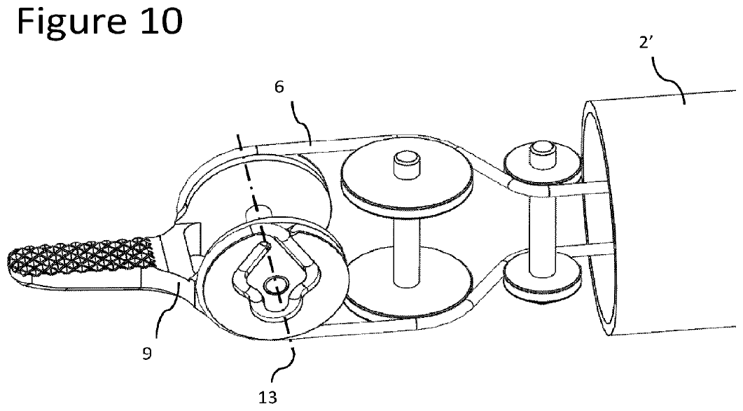

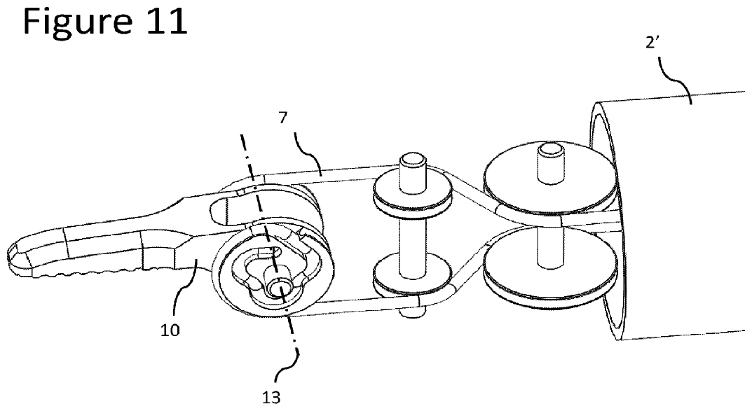

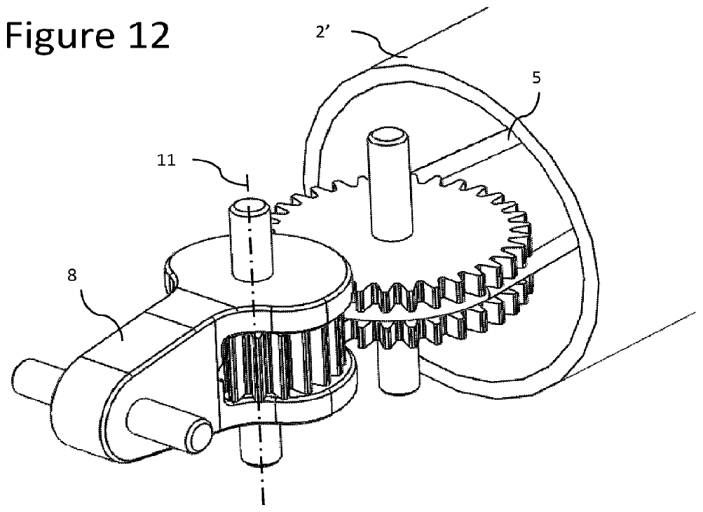

Referring still to FIG. 4, the end-effector 3 is connected to the internal structural element 2″ by a proximal joint, which allows the rotation of the proximal end-effector link 8 about the proximal axis 11 in such a manner that the orientation of the proximal end-effector link 8 with respect to the main shaft axis 12 can be changed. The distal end-effector links 9, 10 are rotatably connected to the proximal end-effector link 8 by two distal joints, having coincident axes of rotation, which are represented by the distal axis 13. This distal axis 13 is substantially perpendicular and non-intersecting with the proximal axis 11 and substantially intersects the main shaft axis 12. FIGS. 5 to 9 show the surgical instrument 1 with different angular displacements at the end-effector joints FIGS. 10 to 12 show the connection between the transmission element 5, 6, 7 and the end-effector links 8, 9, 10.

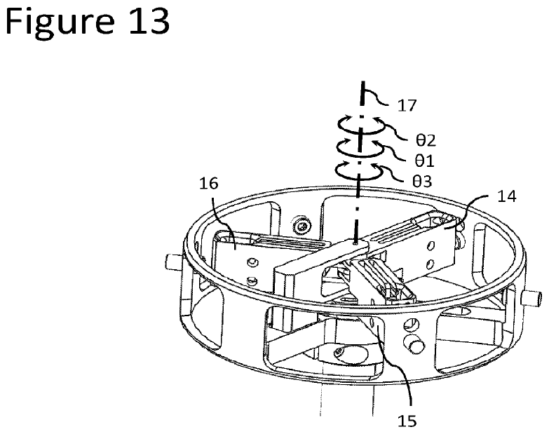

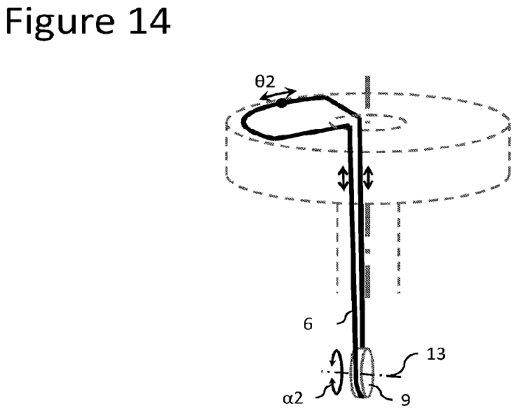

With reference to FIGS. 13 and 14, the movement is transmitted to each one of the three distal articulations of the instrument 1 by a rotating element 14, 15, 16, which is able to rotate about an axis 17 and is connected to a transmission element 5, 6, 7. As a result, when the rotating element 14, 15, 16 rotates a certain angle θ1, θ2, θ3 about the axis 17, a rotation a1, a2, a3 is transmitted to the respective end-effector member 8, 9, 10.

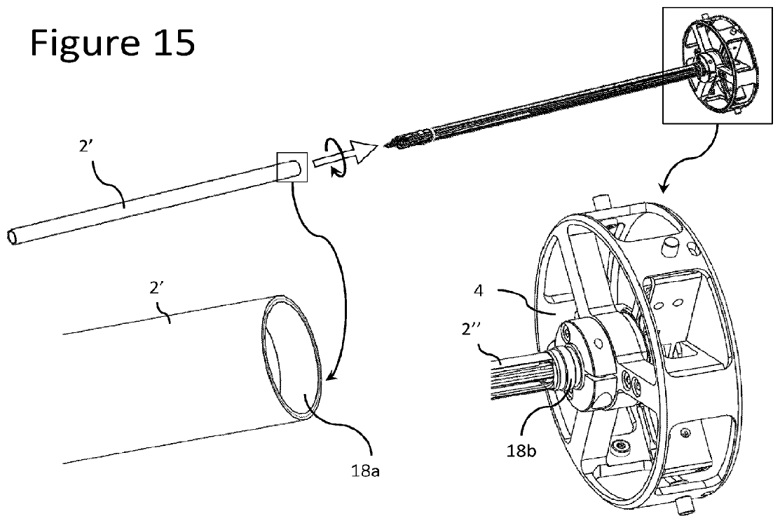

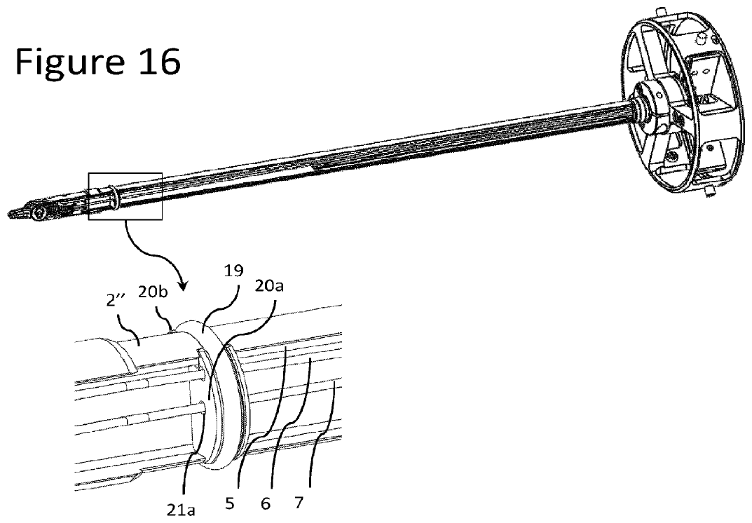

The external tube 2′ can be easily and individually detached and attached to the instrument 1 after each procedure. Referring to FIG. 15, the internal structural element 2″ is fixed directly to the proximal hub 4 and the external tube 2′ can be connected and disconnected from the internal structural element 2″ at the threaded surfaces 18a and 18b. Therefore, with this architecture, the external tube 2′ can be removed from the instrument 1, without the need to disassemble other parts of the system, like the articulated end-effector 3 or the mechanical transmission elements 5, 6, 7, which remain completely operational from a mechanical perspective without the external tube 2′. This feature facilitates the effective cleaning and sterilization of the instrument 1, which can easily be performed by the hospital staff.

Towards a more distal region of the instrument shaft 2, the external tube 2′ is in contact with a sealing element 19, which fills the gap between the internal surface of the external tube 2′ and the two transversal elements 20a, 20b that are mounted on the internal structural element 2″. These two transversal elements 20a, 20b have small channels 21a, 21b, 21c, 21d, 21e, 21f through which the transmission elements 5, 6, 7 can pass, guaranteeing the air-tightness of the instrument 1.

In some embodiments of the present invention, the mechanical transmission elements 5, 6, 7 may comprise ropes, whose tension can be released after each procedure, so that the cleaning and sterilization procedures become easier. By releasing the tension on the ropes, the blood and tissue infiltrated amongst the strands of the ropes can be more easily removed. In addition, areas of contact between the ropes and other mechanical elements (like pulleys, end-effector links 8, 9, 10 or rotating elements 14, 15, 16) can be more easily accessed.

While this invention has been shown and described with reference to particular embodiments thereof, it will be understood by those skilled in the art that various changes in form and details may be made therein without departing from the spirit and scope of the invention as defined by the appended claims. For instance, the external tube 2′ can be made out of different parts and can be attached to the proximal hub 4. In another embodiment, the internal structural element 2″ can also be composed of different parts and can assume different geometries with diverse cross sections, namely tubular (with openings) or U-shaped.

It will also be easily understood by one of skill in the art that the invention can easily be deployed in the context of other micro-manipulation tasks where complex instruments are used, but regular cleaning and/or sterilization of internal elements of an instrument shaft is desirable. Solely by way of example, micro-manipulation tasks are performed in contaminated environments, wherein thorough cleaning of instrument elements is required after each use. In this context, a detachable outer shaft allowing access to internal elements may be desirable.

1.-18. (canceled)

19. A reusable surgical instrument, comprising:
an articulated end-effector, mounted on a distal extremity of the reusable surgical instrument, comprising one or more end-effector links;
a proximal hub;
mechanical transmission elements configured to transmit motion from the proximal hub to the articulated end-effector;
a shaft which defines the longitudinal axis of the instrument, comprising one or more internal structural elements incorporating the mechanical transmission elements;
an external tube configured to protect the internal structural elements and prevent the passage of air through the reusable instrument; and
at least one transversal element is mounted on the one or more internal structural elements to improve an air-tightness of the reusable instrument that prevents a passage of air through the reusable instrument during operation.

20. The reusable surgical instrument of claim 19, wherein the at least one transversal element comprises one or more small channels through which the mechanical transmission elements can pass.

21. The reusable surgical instrument of claim 19, wherein the at least one transversal element is in contact with one or more of a sealing element and the external tube.

22. The reusable surgical instrument of claim 21, wherein the sealing element is configured to fill a gap between an internal surface of the external tube and the at least one transversal element.

23. The reusable surgical instrument of claim 19, wherein the at least one transversal element mounted on a distal region of the shaft.

24. The reusable surgical instrument of claim 19, wherein the at least one transversal element is configured to maintain insufflation of the body cavity.

25. The reusable surgical instrument of claim 19, wherein the external tube is configured to maintain insufflation of the body cavity.

26. The reusable surgical instrument according to claim 19, wherein the mechanical transmission elements comprise flexible mechanical elements, selected from the group consisting of wires, chains, ropes and belts.

27. The reusable surgical instrument of claim 19, wherein a tension on the mechanical transmission elements can be released after use to facilitate effective cleaning and sterilization procedures.

28. The reusable surgical instrument of claim 19, wherein the proximal hub is configured to be mounted on a proximal extremity of the reusable instrument.

29. The reusable surgical instrument of claim 19, wherein the proximal hub comprises one or more rotating elements configured to actuate the end effector links.

30. The reusable surgical instrument of claim 28, wherein the proximal hub is configured to be attached to a mechanical platform comprising a master-slave telemanipulator, such that motion is transmitted from a proximal articulated handle of the master-slave telemanipulator to the proximal hub.

31. The reusable surgical instrument of claim 30, wherein the proximal extremity is configured to releasably couple to the mechanical platform.

32. The reusable surgical instrument of claim 19, wherein the internal structural element is configured to provide stable positioning to the articulated end-effector.

33. The reusable surgical instrument of claim 19, wherein the shaft is configured to position the end-effector within an interior of the patient's body, allow the passage of the different transmission elements that are able to actuate the different distal end-effector articulations, and avoid the passage of air through the instrument, in order to maintain the inflation of the body cavity where the instrument is operating.

34. The reusable surgical instrument of claim 19, wherein the reusable surgical instrument may be configured to be cleaned and sterilized after each procedure.

35. The reusable surgical instrument of claim 19, wherein the reusable surgical instrument comprises one or more of a dissector, a scissor, and a grasper.

36. The reusable surgical instrument of claim 19, wherein motion is transmitted from the proximal hub to the articulated end-effector via the mechanical transmission elements from a set of actuation elements.

37. The reusable surgical instrument of claim 34, wherein the set of actuation elements are located at a proximal extremity of the shaft.
